(19) United States
(12) Patent Application Publication
(10) Pub. No.: US 2006/0099673 A1
(43) Pub. Date: May 11, 2006

(54) Novel recombinant gene expression method by stop codon suppression

(76) Inventors: Hansjorg Hauser, Braunschweig (DE); Peter P. Muller, Salzgitter-Thiede (DE); Klaus P. Schafer, Konstanz (DE); Wolfram Steinhilber, Stockach (DE)

Correspondence Address: NATH & ASSOCIATES PLLC, 112 South West Street, Alexandria, VA, 22314, US

(21) Appl. No.: 10/485,397

(22) PCT Filed: Jul. 31, 2002
(86) PCT No.: PCT/EP02/08520

§ 371 Date: Jan. 30, 2004

(30) Foreign Application Priority Data
Aug. 2, 2001 (EP) 0118632.7

(51) Int. Cl. C07H 21/04; C12P 21/06; C07K 14/47

(52) U.S. Cl. 435/69.1; 435/320.1; 435/325; 530/350; 536/23.5

(57) ABSTRACT

This invention describes a novel recombinant gene expression method based on a novel recombinant gene expression vector, comprising in the following order a promoter sequence, a gene of interest, a translational stop signal and translationally linked to said gene of interest a selectable marker gene.

### TECHNICAL FIELD

The present invention relates to the field of recombinant protein expression in host cells, to a new method to obtain and identify cell clones expressing a gene of interest above a threshold level and stably maintaining or increasing expression levels during cultivation and to a process for producing recombinant proteins.

### PRIOR ART

The general principle of genetic modification of cultured cells or whole animals by various gene transfer methods is well known and a key strategy in both basic research and biotechnological applications. For large scale productions and also for many small scale applications efficient and stable expression of a recombinant product is an absolute requirement. However, difficulties exist in identifying host cells that produce recombinant protein at sufficient levels and in identifying host cell clones that maintain expression levels during prolonged cultivation periods.

Many approaches focusing on recombinant gene expression require co-expression of different gene products in a modified host cell. Frequently used procedures to generate a modified host cell producing a specific protein demand the introduction of foreign nucleic acid, e.g. an expression vector, into said host cell and the simultaneous expression of a selectable marker gene and a gene of interest. This is frequently achieved by co-transferring both genes on a separate DNA construct or alternatively by placing both gene expression cassettes on a single vector provided with two heterologous sets of transcriptional regulatory elements (e.g. pCDNA3 in Huang, C. F. et al., (2001) Disrupting the transforming activity of shrimp ras(Q(61)K) by deleting the CAAX box at the C-terminus. J. Exp. Zool. 289: 441-448). Frequently, the majority of clones that survive the selection process do not express a protein of interest above a threshold level. Usually below 1% of selected clones are high level producers [Fussenegger, M. et al., (1999) Genetic optimization of recombinant glycoprotein production by mammalian cells. Trends in Biotechnology 17: 35-42]. Both approaches rarely result in the generation of cell lines that express the desired gene product over extended periods of time. Cell clones that express the product at moderate levels under selection pressure are frequently used for experimental purposes, but these cell clones do not meet industrial requirements. To obtain stable cell clones time consuming and laborious screening is necessary. The lack of expression stability using the above described gene expression methods is a consequence of the independent expression levels of a selectable marker gene and a gene of interest, i.e. a high selectable marker gene expression level in a given cell clone does not correlate with a high co-expression of said gene of interest. This situation may be improved by using constructs in which both genes are transcribed from the same promoter, i.e. polycistronic expression constructs or bidirectional promoters.

EP-A-0117058 describes an expression vector capable of expressing a desired protein in a vertebrate host cell, which vector comprises a first DNA sequence encoding for a desired protein and a second DNA sequence encoding for a screening marker protein where both said DNA sequences are operationally linked to the same promoter sequence and separated by a translational stop and a start codon; said genes are not translationally linked. Consequently, both proteins are produced separately in mature form.

WO 8805466 describes compositions and methods for expressing gene products of interest in eukaryotic cells with a recombinant dicistronic DNA expression vector that comprises a gene of interest and a selectable marker gene wherein the sequences are physically linked to one another and depend on one common promotor. Thus a cell transfected by such a vector is capable of expressing the gene products from both of the genes.

WO 9708330 is related to new expression systems and in particular to an expression system in which a gene of interest is expressed at an optimal level. The invention provides a recombinant expression vector comprising a gene of interest and a selectable marker gene, wherein the selectable marker gene is arranged downstream of the gene of interest and a stop codon associated with the gene of interest is spaced from the start codon of said selectable marker gene at a distance which is sufficient to ensure that translation reinitiation is required before said selectable marker protein is expressed from corresponding mRNA. Both genes are not translationally linked.

Importantly, the above mentioned examples describe the presence of a translation initiation codon for the selection marker that is outside of the reading frame of the gene of interest. Increasing the number of in-frame stop codons terminating the gene of interest does not invariably lead to a reduced translation of the selection marker reading frame. The reading frames of the gene of interest and the selectable marker gene do not overlap and are not in a defined relationship.

WO0144516 describes a high throughput method of assaying for compounds that inhibit premature translation termination and nonsense mediated RNA decay in cells. The method described is based on the use of nucleic acid encoding a polypeptide, wherein the coding sequence for the polypeptide comprises a premature stop codon.

Zinoni et al. [Zinoni F et al. (1990) PNAS 87: 4660] describe fdhF-lacZ fusion constructs to analyse the discrimination of the internal fdhF-mRNA UGA codon. According to table 2, fdhF-lacZ constructs comprise the following cassette: 5′-part of lacZ−fdhF+internal UGA−3′-part of lacZ—stop codon.

Sogaard et al. [Sogaard T M M et al. (1999) Biochemistry 64: 1668] describe an in vivo system to monitor translation termination activity. The system is based on heat stable secreted alkaline phosphatase (SEAP) where suppression at the stop codon tags said protein with an S-peptide. The fusion protein comprising S-peptide specifically binds to S-protein which is coated on microtiter plates. Comparison of SEAP activity in the supernatant with SEAP-activity coated on the microtiter plate is a mean to monitor in vivo translation termination.

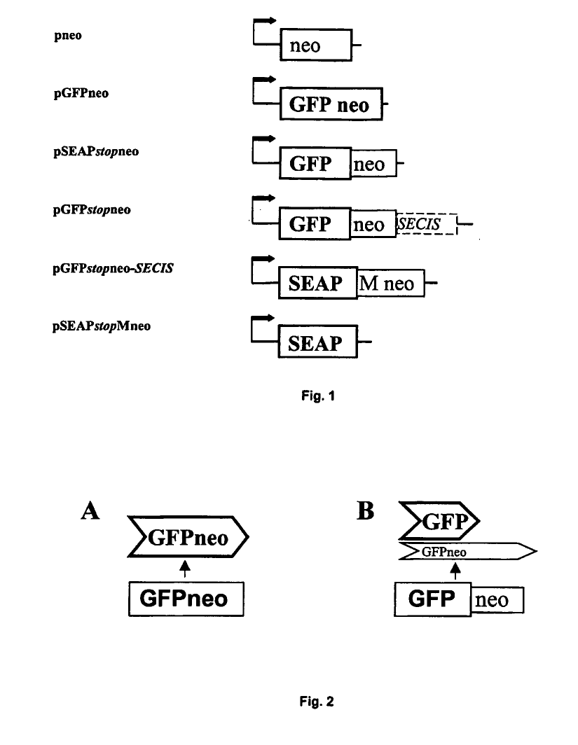

Goldman et al. [Goldman E et al. (2000) FASEB 14: 603] describe the cloning of an unusual nucleotide sequence, H10, into a reporter system (using β-galactosidase) and the testing for expression in E. coli. FIG. 1 discloses the experimental design. DNA constructs comprise in the following order a start for transcription, FLAG, H10 sequence comprising internal stop codons, an E-tag and a gene encoding β-galactosidase. Goldman et al. do not disclose a recombinant gene expression vector as disclosed herein and do also not describe a method to isolate stable expressing bacterial and mammalian cell clones.

Danielson et al. [Danielson S et al. (2001) Gene 272: 267] describe the insertion of the wild type lipolase gene into a phagemid vector. The resulting construct encodes for the secreted expression of lipase, as fused to a 5 kDa serum albumin binding domain, followed by a suppressable TAG stop codon and a truncated version of the M13 phage coat protein 3. It also describes the expression of the lipase as a lipase-ABD fusion protein. Danielson et al. do not describe DNA constructs as disclosed herein and do not disdose a method to isolate stable expressing bacterial and mammalian cell clones.

Kollmus et al. [Kollmus H. et al., (1996) Analysis of eukaryotic mRNA structures directing cotranslational incorporation of selenocysteine. Nuleic Acids Research 24:1195-1201] describe an assay system designated to facilitate analysis of DNA elements that influence stop codon suppression in mammalian cells. It is based on reporter genes encoding β-galactosidase and luciferase, which are fused in frame via a TGA stop codon. The DNA is transcribed under control of the SV40 promoter and translation leads to the synthesis of said reporter enzymes generating either α-galactosidase or, upon suppression of the TGA codon, a fusion protein of β-galactosidase and luciferase. Kollmus et al. do not describe a recombinant gene expression vector comprising a selectable marker gene, as specified according to this invention. In addition, Kollmus et al. do not disclose a method to isolate stable expressing bacterial and mammalian cell clones.

None of the above cited patent or literature documents does describe a process of producing a host cell line which cell line efficiently expresses a gene of interest.

In summary, even with polycistronic constructs, high selection pressure or prolonged cultivation of cells result in the isolation of cell clones that efficiently express the selectable marker gene but not the gene of interest. Therefore, it is a considerable problem that there is no reliable and for the average research laboratory affordable method to isolate stable expressing bacterial and mammalian cell clones that express a gene of interest at a high level.

### DESCRIPTION OF THE INVENTION

This invention describes a novel recombinant gene expression method based on a novel recombinant gene expression vector comprising a gene encoding a selectable marker protein which is separated by a translational stop signal from an upstream arranged gene of interest, whereby both genes are translationally linked. Consequently, the expression of said selectable marker gene may be reduced compared to the expression rate of said gene of interest.

Surprisingly, it has now been found that a high proportion of expressing single cell clones may be obtained under selection pressure by the usage of said novel recombinant gene expression vector according to this invention. In said expressing single cell clones the stop codon-dependent translational coupling of marker gene expression with gene of interest expression results in two recombinant gene products, i.e. a product encoded by the gene of interest and as a fusion protein the gene of interest combined with the selectable marker gene.

More surprisingly, the novel recombinant gene expression vector can be used to achieve stable gene expression under selection conditions by resisting cellular mechanisms that uncouple the resistance to selection conditions from the expression of a recombinant gene of interest. Thus, the expression of the gene of interest is enhanced or stabilized by selection for activity of the encoded selectable marker protein.

In particular surprisingly, it was also found that stop codon suppression could be applied to reach selection marker gene expression levels and selection marker activity sufficient to achieve resistance of transfected cells to the selection procedure. It is even more particularly surprising that said cell clones comprising said novel recombinant gene expression vector and expressing said selection marker gene at levels sufficient to achieve resistance required for cell survival under selective conditions express said gene of interest above a threshold level, whereby said level is at least identical or superior to the yields generated by cell clones comprising a standard recombinant gene expression vector and expressing a gene of interest based on standard techniques.

It is surprising that a host cell comprising a recombinant gene expression vector as specified herein comprising both a translational stop signal and a gene of interest encoding a secreted product protein is capable in producing a homogenous product of said gene of interest whereby the amount of homogenous gene product produced is identical or superior to the yields generated by host cells comprising a standard recombinant gene expression vector comprising a gene encoding a secreted product protein.

The advantage of this system is an extremely tight coupling of selectable marker gene expression and gene of interest expression. This property results in several advantageous features as follows:
Increased frequency of transformed/transfected host cells expressing said gene of interest after selection for cells expressing the selectable marker gene.
By limiting the expression of the selectable marker gene at the translational level, transformed/transfected host cells with an increased transcriptional activity of the recombinant genes are selected. Thus the average recombinant protein expression of the gene of interest is also increased when compared to expression vectors without limiting selection gene expression.
Increased stability of gene of interest expression under selective conditions when compared to conventional expression vectors with other mechanisms of selection, such as polycistronic vectors [Mueller, P. P. et al. (1999) Recombinant glycoprotein product quality in proliferation controlled BHK-21 cells. Biotech. and Bioeng. 65: 529-536] or vectors with bidirectional promoters [Baron, U. et al. (1995) Co-regulation of two gene activities by tetracycline via a bidirectional promoter. Nucleic Acids Research 23: 3605-3606].

In a first embodiment of the invention there is provided a recombinant gene expression vector comprising in the following order a promoter sequence, a gene of interest, a translational stop signal and translationally linked to said gene of interest a selectable marker gene, whereby a recombinant gene expression vector is excluded which recombinant gene expression vector contains in the following order a promoter sequence, a gene encoding β-galactosidase, a TGA stop codon, and translationally linked to said β-galactosidase gene a gene encoding luciferase.

In a second embodiment of this invention there is provided a recombinant gene expression vector as disclosed before comprising more than one gene of interest.

Preferably, the present invention provides a recombinant gene expression vector comprising said elements in said order whereby the gene of interest and the selectable marker gene are translationally linked. In accordance to this invention, translationally linked refers to a reading frame fusion, such that ribosomes translate the selection marker from the same translation initiation site as the gene of interest. It is particularly preferred that translationally linked refers to constructs wherein the gene of interest and the selectable marker gene are entirely or partially in the same reading frame. The gene of interest and the selectable marker gene may be separated either by a translational stop signal and/or translational frameshifting signals.

This invention refers to any sequence of desoxyribonuleotides which result in (1) an in frame arrangement of said gene of interest and said selectable marker gene and (2) a mRNA product comprising both said gene of interest and said selectable marker gene. Thereby, the gene of interest is located at the 5′-end of the reading frame fusion and it may be translated independently of the downstream arranged selectable marker gene. In addition, it is an arrangement whereby the reading frame of said selectable marker gene is arranged downstream of said gene of interest in a way that the translation of the selectable marker gene reading frame is dependent on prior translation of the gene of interest such that the selectable marker gene product may be expressed as a fusion protein. In particular, the invention relates to said recombinant gene expression vector in a way that a separate translational start codon for the selectable marker gene reading frame may be omitted.

According to this invention, a gene refers to any sequence of desoxyribonucleotides encoding the information for the synthesis of a primary RNA transcript or a protein. A gene of interest refers to any nucleic acid sequence used for cloning in said recombinant gene expression vector encoding a protein or protein fragment thereof whose expression is of interest. According to this invention, a gene of interest refers to a wildtype or a modified gene, whereby a wild-type gene refers to any gene that predominates in the population because it confers the greatest fitness (ability to survive and reproduce) and whereby a modified gene refers to any gene which sequence differs from that of the wildtype gene either by mutation, deletion, insertion of a single or multiple nucleotide sequences or by fusion of additional sequence to at least one end of the gene sequence. In a preferred embodiment of this invention, a gene of interest refers to a gene encoding a protein of pharmaceutical interest such as (1) biotechnological products such as cytokines (Insulin, EPO (erythropoietin), TPO (Tissue Plasminogen Activator), GCSF (Granulocyte Colony Stimulating Factor), GMCSF (Granulocyte-Macrophage Colony Stimulating Factor), blood clotting factors FVIII or FVII), (2) other pharmaceutically active products such as surfactant protein (SP) selected from the group of SP-A, SP-B, SP-C or SP-D, (3) gene products used for identification of drug targets, (4) any EST (Expressed Sequence Tag) reading frame, and (5) any reporter gene, in particular SEAP (Secreted Alkaline Phosphatase), lacZ (8-Galactosidase), luc (firefly or renilla luciferase), GFP (Green Fluorescence Protein) and derivatives thereof and proteins with similar function such as Red Fluorescence Protein (RFP), CAT (Chloramphenicol Acetyl Transferase), proteins and reporter protein tags exposed at the cell surface such as myc tag, polyhistidine tag, or flag antigen. In a particularly preferred embodiment of the present invention, said gene of interest preferably encodes a pharmaceutically active protein. A pharmaceutically active protein is understood as any protein capable for usage in diagnosis, prophylaxis, treatment or modification of physiological functions in human or animal body.

In the present invention, one promoter controls the expression of at least one gene of interest and a selectable marker gene. A promoter refers to any DNA sequences involved in promoting transcription of a nucleic acid sequence. In particular, a promoter refers to LTR (Long Terminal Repeat) promoter elements of Retrovirus such as MMTV (Mouse Mammary Tumor Virus), RSV (Rous Sarcoma Virus), MPSV (Myelo-Proliferative Sarcoma Virus). Particularly, a promoter refers to other viral promoters such as SV40 (Simian Virus) early or late promoter, or CMV (CytoMegaloVirus) promoter. It also refers to the EF (Elongation Factor) promoter or to an artificial promoter such as tetracycline regulated promoter [Gossen, M. and Bujard, H. (1992) Tight control of gene expression in mammalian cells by tetracycline-responsive promoters. PNAS 89: 5547-5551], or to any other known promoter, such as eukaryotic, prokaryotic or viral promoters.

The vector used in the process of the invention may be any of the known types, comprising any DNA or RNA segment that can replicate autonomously within a cell, in particular in a bacterial cell, e.g. expression plasmid or viral vectors. In another embodiment of this invention, a vector refers to any DNA segment that can not replicate autonomously within a cell and which DNA segment integrates into the chromosome of said cell.

In a third embodiment of the invention, there is provided a recombinant gene expression vector comprising in the following order a promoter sequence, a gene of interest, a translational stop signal and translationally linked to said gene of interest a selectable marker gene encoding a functional protein, whereby a recombinant gene expression vector is excluded which recombinant gene expression vector contains in the following order a promoter sequence, a gene encoding β-galactosidase, a TGA stop codon, and translationally linked to said β-galactosidase gene a gene encoding luciferase.

Usually, bacterial cells transformed with a plasmid vector are positively selected for dominant antibiotic resistance markers carried by said plasmid, effectively maintaining a population of plasmid-containing cells. The same or alternative markers are used in transfected eukaryotic systems, e.g. neomycin phosphotransferase. Other selection or screening markers can be used if the activity of the fusion gene is high enough to allow marker activity above background levels.

This invention also refers to a recombinant gene expression vector, wherein said selectable marker gene for selection of transfected cells is a drug resistance gene encoding a protein that confers resistance to selection conditions, e.g. neo (neomycin phosphotransferase), hyg (hygromycin acetyltransferase), tk (Herpes simplex thymidine kinase), PAC (pyromycin acetyltransferase), zeo (zeozin resistance gene), DHFR (dihydrofolate reductase).

In a variant of this invention, said selectable marker gene refers to a reporter gene, in particular to GFP (Green Fluorescence Protein), whose expression can be used to differentiate between host cells producing said selectable marker gene and those which do not express said gene.

In particular, the invention refers to a recombinant gene expression vector comprising a selectable marker gene encoding neomycin phosphotransferase. A transfected host cell line comprising this vector may be positively selected from a plethora of cells lacking said vector by using G418. G418 is an aminoglycoside antibiotic that bind to the ribosome and reduce the fidelity of translation. G418 distinguishes between prokaryotic and eukaryotic ribosomes. G418 is a substrate for neomycin phosphotransferase encoded by the bacterial neo gene, resulting in the phosphorylation and thereby the inactivation of G418 [Beck, E. et al. (1982) Nucleotide sequence and exact localization of the neomycin phosphotransferase gene from transposon Tn5. Gene 19: 327-336; Colbere-Garapin, F. et al. (1981) A new dominant hybrid selective marker for higher eukaryotic cells J. Mol. Biol. 150: 1-13].

In a fourth embodiment of this invention there is provided a recombinant gene expression vector as disclosed before, wherein said translational stop signal is at least one stop codon selected from the group of TAA, TGA and TAG.

According to this invention, said selectable marker gene is arranged downstream of said gene of interest, whereby both genes are separated by a translational stop signal. A translational stop signal refers to any genetically element suitable to terminate the translation of a reading frame and thereby decrease the rate of translation of any reading frame encoded in the mRNA downstream of said translational stop signal. In particular, a translational stop signal refers to at least one stop codon on mRNA selected from the group of UGA, UAA and UAG, encoded by TGA, TAA and TAG, respectively, on the corresponding DNA construct. In another embodiment of this invention, more than one stop codon, preferably two or three similar or different stop codons are selected from said group. Consequently, the expression of said selectable marker gene is reduced when compared to a conventional translation fusion whereby the stop codon of the upstream gene is removed. In the absence of additional genetic elements, the reduction of the selection marker gene expression is defined as the natural rate of stop codon read-through. In mammalian cells, the naturally occurring stop codon read-through efficiency is about 0,1% [Kollmus, H. et al. (1996) Analysis of eukaryotic mRNA structures directing cotranslational incorporation of selenocysteine. Nuleic Acids Research 24:1195-1201]. By using several stop codons in frame the naturally stop codon-read-through efficiency can be further reduced or enhanced, respectively.

The read-through efficiency may be modulated by including a stop codon suppression mechanism. This can be a SECIS element for UGA suppression, suppressor tRNAs, ectopic expression of wild-type tRNAs, use of alternative stop codons UAG or UAA, by changing the context of the stop codon, by introducing a frameshift, or by the addition of an alternative transcriptional stop signal. An alternative transcriptional stop signal can be a natural cleavage and poly(A) addition site or a poly(A) consensus sequence inserted preferably downstream of the stop codon of the gene of interest, or in the 5′-region of the selection marker gene [Minvielle-Sebastia, L., Keller, W. (1999) mRNA polyadenylation and its coupling to other RNA processing reactions and to transcription. Curr. Opin. Cell. Biol. 11: 352-357; Wahle, E., Ruegsegger, U. (1999) 3′-End processing of pre-mRNA in eukaryotes. FEMS Microbiol Rev 23: 277-295; Edwalds-Gilbert, G., Veraldi, K. L., Milcarek, C. (1997) Alternative poly(A) site selection in complex transcription units: means to an end? Nucleic Acids Research 25: 2547-2561].

In a fifth embodiment of this invention, there is provided a recombinant gene expression vector comprising a promoter sequence, a gene of interest, a translational stop signal, a selectable marker gene (arranged in frame) and a SECIS element. The SECIS element (selenocystein insertion sequence) is a structural element (hairpin structure) in eukaryotic mRNA that enables the insertion of the 21^{st} amino acid, selenocystein, at sites of the stop codon UGA [Kollmus H et al. (1996) Analysis of eukaryotic mRNA structures directing cotranslational incorporation of selenocysteine. Nuleic Acids Research 24: 1195-1201; Fagegaltier, D. et al. (2000) Structural analysis of new local features in SECIS RNA hairpins. Nucleic Acids Research 28: 2679-2689] and therefore increases the stop codon-read-through and the expression of any downstream localized genes.

Kollmus et al. measured the reduction in stop codon suppression efficiency in BHK-21 cells using the pig heart phospholipid hydroperoxidase glutathione peroxidase (PHGPx) gene. This SECIS element supported a stop codon suppression activity of 2,8%. A minimum SECIS element supported a 10-fold reduced activity of 0,3% stop codon-read-through [Kollmus, H. et al. (1996) Analysis of eukaryotic mRNA structures directing cotranslational incorporation of selenocysteine. Nuleic Acids Research 24: 1195-1201]. The SECIS element of the gene encoding the rat 5′-deiodinase results in a relative stop codon suppression efficacy of 1.1% [Kollmus H et al. (1996) Analysis of eukaryotic mRNA structures directing cotranslational incorporation of selenocysteine. Nuleic Acids Research 24: 1195-1201]. A mutation in this element reduces the efficiency to 0,16%.

According to Kollmus et al. [Kollmus, H. et al. (1996) Analysis of eukaryotic mRNA structures directing cotranslational incorporation of selenocysteine. Nuleic Acids Research 24: 1195-1201], the stop codon suppression efficiency can be calculated by relating expression activity of luciferase—determined by measuring the enzymatic activities of luciferase—of cells containing certain bacterial plasmids to values obtained with a control plasmid. Based on this enzymatic method to determine the stop codon suppression efficiency, a stop codon suppression efficiency of 0,01% to 10% is a preferred embodiment of this invention. In particular, this invention refers to a stop codon suppression efficiency of 0,1% to 5%.

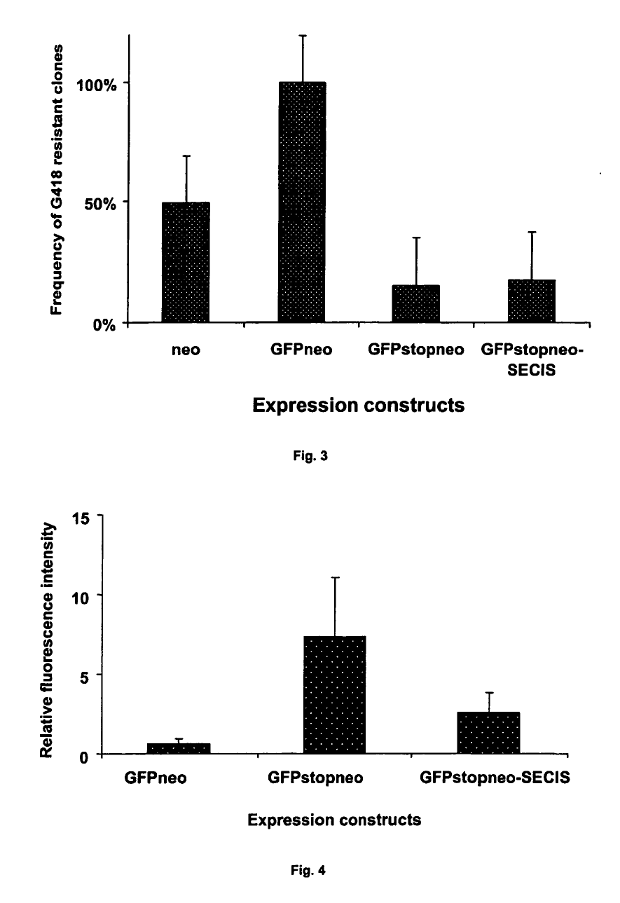

In said recombinant expression vectors, said gene of interest and said selectable marker gene are translationally linked. Therefore, a read-through results in the production of a fusion protein comprising both products of said gene of interest and said selectable marker gene. The function of a protein encoded by said selectable marker gene is conserved within the fusion protein, as exemplified by its ability demonstrated herein to confer antibiotic resistance to cells transfected with these constructs (see FIG. 3). In that way, host cells comprising said recombinant expression vector may be selected by positive selection on expression of said fusion protein.

In a further embodiment of this invention, there is provided a host cell comprising a recombinant gene expression vector as disclosed herein which host cell is capable of expressing both said gene of interest and as a fusion protein said gene of interest and said selectable marker gene.

According to this invention, a host cell comprising a recombinant gene expression vector as disclosed herein comprises a eukaryotic or a prokaryotic cell line transfected/transformed with said recombinant gene expression vector.

According to this invention, transformation is synonymous with transfection.

According to this invention, a eukaryotic host cell line comprising said recombinant gene expression vector refers to any type of cell comprising a cell culture cell such as BHK-21, CHO (various Chinese Hamster Ovary cell lines), 293 (human kidney carcinoma cell line), NIH 3T3 (murine cell lines), SP2/0 (hybridoma), other eukaryotic cell lines of pharmaceutical or laboratory research interest, primary cells such as ES cells (Embryonic Stem cells), other mammalian stem or progenitor cells such as bone marrow progenitor cells, putting together a whole organism, whereby said recombinant gene expression vector has been transferred into said host cell line by transfection. According to this invention, transfection refers to (1) the techniques specified in Sambrook et al. [Sambrook et al. (1989) In: Molecular Cloning: A Laboratory Manual. Cold Spring Harbor Laboratory Press, second edition], e.g. calciumphosphate-transfection, transfection using DEAE (Diethylaminoethyl)-dextran, lipofection, (2) electroporation and (3) infection, e.g. via a viral vector system. In another variant of this invention, said process of transfecting a host cell line comprises specific techniques. In particular, specific techniques refer to techniques revealed by following publications: Wigler, M. et al. (1977) Transfer of purified herpes virus thymidine kinase gene to cultured mouse cells. Cell 11: 223-232; Chesnoy S and Huang L (2000) Structure and function of lipid-DNA complexes for gene delivery. Annu. Rev. Biophys. Biomol. Struct. 29: 27-47; De Smedt S C, Demeester, J., Hennink, W. E., (2000) Cationic polymer based gene delivery systems. Pharm. Research 17: 113-26; Mahato, R. I. (1999) Non-viral peptide-based approaches to gene delivery. J. Drug Target 7: 249-268; Stone, D. et al. (2000) Viral vectors for gene delivery and gene therapy within the endocrine system. J. Endocrinol 164: 103-118; Zhao, X., (2000) Gene transfer and drug delivery by electronic pulse delivery. A nonviral delivery approach. Methods Mol. Biol. 133: 37-43. As an alternative, other methods not known to a person skilled in the art can be used. For selection of transfected cells, methods described in Sambrook et al. [Sambrook et al. (1989) In: Molecular Cloning: A Laboratory Manual. Cold Spring Harbor Laboratory Press, second edition], or methods specified in the above mentioned publications concerning transfection of eukaryotic cells and other selection methods known to a person skilled in the art can be used.

In the case of a bacterial host cell the transformation and selection of positive clones may be performed according to the techniques specified in Sambrook et al. [Sambrook et al. (1989) In: Molecular Cloning: A Laboratory Manual. Cold Spring Harbor Laboratory Press, second edition] or by electroporation, bacteriophage infection or other methods known to a person skilled in the art.

Said transformation methods may be used by using said expression vector and selecting a transformed/transfected host cell on the product expression of said selectable marker gene. This process refers to both prokaryotic and eukaryotic cells. More preferred, this process refers to cells that are unstable or unpredictable in the expression level of recombinant proteins.

In a further embodiment of this invention, there is provided a process for producing a host cell clone comprising a recombinant gene expression vector as disclosed herein, which process comprises transforming a host cell with said recombinant gene expression vector and selecting a host cell clone comprising said recombinant gene expression vector on the expression of said selectable marker gene.

According to this invention, a host cell clone refers to a group of host cells, all of which are derived from a single individual cell through asexual reproduction. Except from changes that come about by mutation, all members of a host cell clone are genetically identical. Continued growth of a host cell clone in a laboratory culture results in a host cell line.

According to this invention a transformed/transfected host cell comprising said recombinant gene expression vector particularly express a sufficient amount of protein encoded by said selectable marker gene being an adequate amount of said selectable marker gene product to result in cell clones that survive the selection conditions after transformation of host cells with said recombinant expression vector under conditions that lead to cell death of comparable cells which do not carry said recombinant gene expression vector. In particular, a sufficient amount of protein encoded by said selectable marker gene refers to an adequate amount of said selectable marker gene product to select at least one transformed host cell out of a plethora of cells after introducing DNA or RNA of said recombinant expression vector into host cells.

In another embodiment of this invention there is provided the use of a recombinant gene expression vector as disclosed herein in a process for efficiently selecting a host cell clone highly expressing said gene of interest.

According to this invention, efficiently selecting refers to a method of transforming host cells, selecting said transformed host cells on the expression of said selectable marker gene—whereby those host cells survive the selection conditions which carry said recombinant expression vector and express said selectable marker gene under conditions that lead to cell death of comparable cells which do not carry said recombinant gene expression vector—and identifying at least 5% of host cell clones highly expressing said gene of interest compared to the total number of host cell clones surviving selection conditions. In particular, efficiently selecting refers to a selection method as mentioned before, whereby at least 10% of host cell clones highly expressing said gene of interest survive selection conditions. It is particularly preferred that efficiently selecting refers to a selection method as mentioned before, whereby at least 14% of host cell clones highly expressing said gene of interest survive selection conditions.

According to this invention, highly expressing said gene of interest refers to expressing a protein encoded by said gene of interest above a threshold level. In the meaning of this invention, a threshold level refers to at least 0,5% of total cell protein. In particular, a threshold refers to at least 5% of total cell protein. It is preferred, that a threshold level refers to at least 25% of total cell protein. It is particularly preferred that a threshold level refers to at least 50% of total cell protein. It is even more preferred that a threshold level refers to at least 75% of total cell protein. In particular, a threshold level of said recombinant expressed protein produced in mammalian cells refers to at least 1 μg protein per 1 ml culture supernatant final concentration. For mammalian cells a threshold level of at least 10 μg/ml is preferred. It is particularly preferred that said threshold level refers to at least 25 μg/ml. It is even more particularly preferred that said threshold level refers to at least 50 μg/ml. Specifically, it is even more particularly preferred that said threshold level refers to at least 100 μg/ml.

In accordance to this invention, protein concentrations are determined according to the methods described by Sambrook et al. [Sambrook et al. (1989) In: Molecular Cloning: A Laboratory Manual. Cold Spring Harbor Laboratory Press, second edition].

In a further embodiment of this invention there is also provided a process of producing a host cell line highly expressing a gene of interest, which process comprises a process for producing a host cell clone as disclosed herein comprising transforming a host cell with said recombinant gene expression vector, selecting a host cell done comprising said gene expression vector on the expression of said selectable marker gene and identifying a host cell clone which highly expresses said gene of interest.

A transformed/transfected host cell comprising said recombinant expression vector is capable of expressing a sufficient amount of protein encoded by said selectable marker gene being an adequate amount of said selectable marker gene product to result in cell clones that survive the selection conditions after transformation of host cells with said recombinant expression vector under conditions that lead to cell death of comparable cells which do not carry said recombinant gene expression vector. In particular, a sufficient amount of protein encoded by said selectable marker gene refers to an adequate amount of said selectable marker gene product to select at least one transformed host cell out of a plethora of cells after introducing DNA or RNA of said recombinant expression vector into host cells.

In a further embodiment of this invention, there is provided a process for producing a protein encoded by a gene of interest, which process comprises the production of a host cell line comprising a recombinant gene expression vector, the cultivation of said host cell line, stably maintaining the expression of said gene of interest, and recovery of the product of said gene of interest, whereby said recombinant gene expression vector comprises in the following order a promoter sequence, a gene of interest, a translational stop signal and translationally linked to said gene of interest a selectable marker gene, and whereby the production of said host cell line comprises the transformation with said recombinant gene expression vector and the selection of a host cell comprising said recombinant gene expression vector on the expression of said selectable marker gene.

According to this invention, stably maintaining the expression of said gene of interest during cultivation refers to approximately constant production of proteins encoded by said gene of interest and said selectable marker gene over the period of 10 passages of the cell line. In particular, 25 cell passages are preferred. It is particularly preferred, that said host cell line produces said proteins at approximately constant level over 50 cell cycles. It is even more particularly preferred, that said host cell line produces said proteins at approximately constant level over 100 cell cycles. In a variant, it is preferred that stably maintaining the expression of said gene of interest during cultivation of transformed bacterial cells refers to recombinant protein productivity in bacteria that are superior to the stability of productivity obtained with related DNA constructs that do not employ the gene fusion technology described herein.

In another embodiment of this invention there is provided a host cell line comprising a recombinant gene expression vector as disclosed herein which host cell line is stably maintaining the expressing of said gene of interest during cultivation.

Most eukaryotic proteins are synthesized in the cytoplasm. Therefore, proteins must carry recognizable sequences or structures which allow them to be transported to the appropriate cellular compartment. Preproteins contain conserved amino acid residues, particularly found at the termini of preproteins (signal peptides) containing the information for cellular transporting. In eukaryotes, proteins destined for secretion, i.e. secreted product proteins, are initially targeted to the endoplasmatic reticulum (ER). This requires a N-terminal hydrophobic sequence signal peptide. The signal sequence is cleaved as the polypeptide is cotranslationally entering the ER lumen. Transmembrane proteins, e.g. placental alkaline phosphatase and many cell surface receptor subunits such as the platelet derived growth factor (PDGF) receptor, possess an internal hydrophobic stop transfer sequence often flanked with positively charged residues [Dalbey, R. E. (1990) Positively charged residues are important determinants of membrane protein topology. Trends Biochem. Sci. 15: 253-257]. The presence of a membrane-spanning domain in a secreted protein retains such a protein in cellular membranes such as the ER membrane, Golgi or cell membrane. Removal of the membrane-spanning domain in such proteins results in the secretion, as it is the case with SEAP, the secreted form of alkaline phosphatase. Membrane proteins of bacterial cells may also have hydrophobic stop transfer sequences to ensure their incorporation into the bacterial membrane.

In a further embodiment, there is also provided a recombinant gene expression vector comprising in the following order a promoter sequence, a gene of interest, a translational stop signal, a stop transfer sequence and translationally linked to the gene of interest a selectable marker gene. Said expression system is applicable to secreted product proteins.

Eukaryotic host cells comprising said recombinant gene expression vector may be positively selected on the expression of said fusion protein, comprising said selectable marker. Fusion proteins may be retained in the ER lumen thereby mediating resistance, whereas a product of said gene of interest is secreted in the cell's surrounding culture medium. This system allows a simple and selective recombinant production of a secreted product protein.

In another embodiment of this invention, there is provided a process for producing a secreted product protein encoded by a gene of interest, which process comprises the production of a host cell line comprising a recombinant gene expression vector, the cultivation of said host cell line, stably maintaining the expression of said gene of interest, and recovery of said secreted product protein from the cell's surrounding culture medium, whereby said recombinant gene expression vector comprises in the following order a promoter sequence, a gene of interest encoding a secreted product protein, a translational stop signal, an in frame stop transfer sequence and translationally linked to said gene of interest a selectable marker gene, and whereby the production of said host cell line comprises the transformation with said recombinant gene expression vector and the selection of a host cell comprising said recombinant gene expression vector on the expression of said selectable marker gene. In a variant of this invention said recombinant gene expression vector comprising said genetic element may also be used for expression of secreted product proteins in bacterial cells.

The invention will now be particularly described by way of examples with reference to the given figures. The following examples do not limit the scope of the invention.

Description of Diagrams:

FIG. 1. Expression constructs used for stop codon suppression dependent expression.

Plasmids were constructed from DNA fragments containing transcriptional units encoding the drug resistance gene neomycin phosphotransferase (neo) or the GFP-neo fusion protein (GFPneo). Plasmids were constructed with a GFP-neo fusion protein separated by an in-frame stop codon (GFPstopneo), with a GFP-neo fusion protein separated by an in-frame stop codon and followed by an element (SECIS) that promotes the insertion of the amino acid selenocystein at the stop codon (GFPstopneo-SECIS), and with a secreted alkaline phosphatase (SEAP) reading frame terminated by a stop codon. The downstream sequences can only be translated when an error occurs and the ribosomes do not terminate translation at the stop codon. SEAP is followed by a membrane spanning domain (M) fused to the neo reading frame (SEAPstopMneo); the membrane spanning domain is derived from the platelet-derived growth factor receptor (PDGFR) isolated from the plasmid pDISPLAY (Invitrogen) and serves the purpose to anchor the secreted protein to the plasma membrane. Thereby the secreted product SEAP is exposed on the extracellular side, while neo protein is on the intracellular face of the membrane. The intracellular localization is considered a prerequisite to confer antibiotic resistance.

FIG. 2. Translation products encoded by the GFPneo and GFPstopneo reading frames.

The GFPneo reading frame (A, square box) encodes a single GFPneo fusion protein (A, upper arrow). The GFPstopneo construct contains the GFP reading frame (that is terminated by an in-frame stop codon) and the neo reading frame. The GFP reading frame encodes the GFP protein (B, upper arrow) and the natural rate of stop codon suppression would result in a small amount of GFP-neo fusion protein (B, lower arrow).

FIG. 3. Frequency of resistant cell clones obtained with stop codon suppression dependent expression constructs.

Equal amounts of plasmid DNA encoding the drug resistance gene neo (neo; GFP-neo fusion (GFPneo); a GFP-neo fusion separated by an in-frame stop codon (GFPstopneo); a GFP-neo fusion separated by an in-frame stop codon followed by a stop codon suppressing element (GFPstopneo-SECIS) were used to stably transfect BHK-21 cells (neo) using the calcium phosphate coprecipitation method (Pellicer, A. et al. (1978) The transfer and stable integration of the HSV thymidine kinase gene into mouse cells. Cell 14: 133-141.). After selection of surviving G418 resistant cells in the presence of 1000 μg/ml of the drug G418 in standard DMEM (Gibco) with 10% fetal calf serum albumin (FCS) at a temperature of culture of 37° C. in a 5% CO₂ incubator, the number of clones was determined for each construct and compared to the number of clones obtained with a separate neo construct (Frequency of resistant cell clones). The bar on top of the columns indicates the standard deviation.

FIG. 4. Gene expression in cell clones with stop codon suppression dependent translation.

BHK-21 cells were stably transfected with the plasmid DNA indicated (see FIG. 1). After selection for neo expressing clones in G418 containing cell culture medium, GFP fluorescence intensity of single suspended cells was determined by Fluorescence Activated Cell Sorter (FACS) analysis.

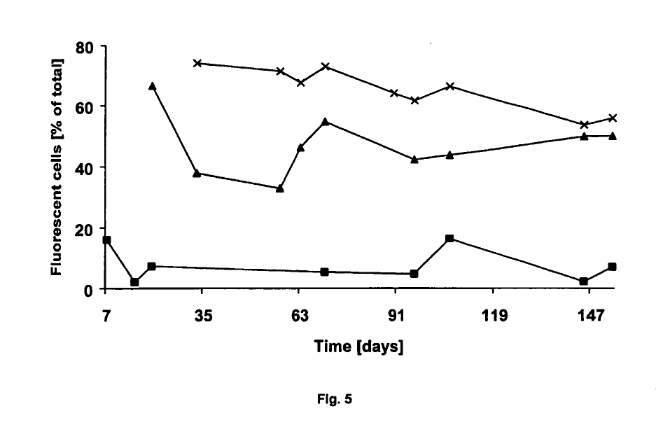

FIG. 5. Stability of expression during cultivation under stop codon suppression dependent selection conditions.

Stability of GFP expression from a GFP-neo reading frame fusion construct separated by an in-frame stop codon (GFPstopneo) was examined during extended cultivation time in 3 different single cell clones relative to a non-fluorescent reference cell clone by FACS analysis of the fluorescence from individual cells. Shown are the percentage of cells with GFP fluorescence above non-GFP-containing cell fluorescence intensity.

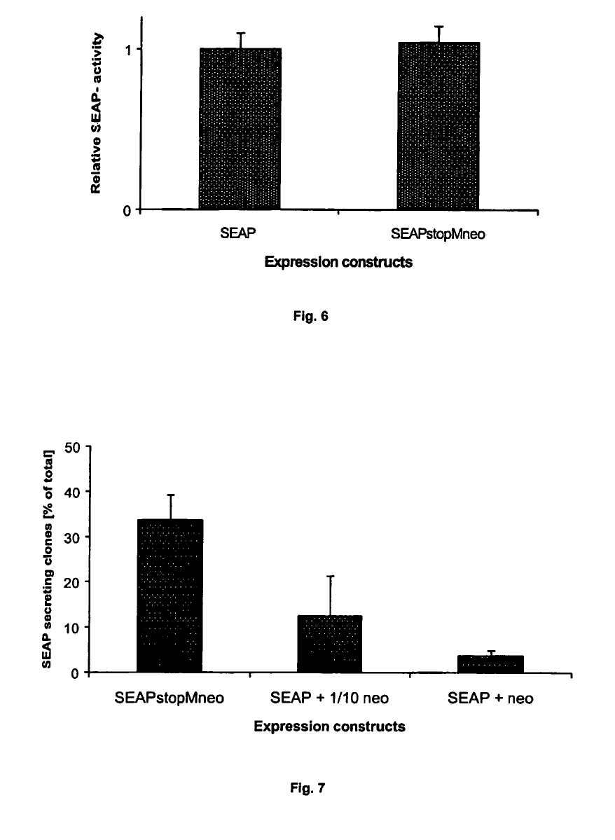

FIG. 6. Secreted protein expression from stop codon suppression dependent translation constructs in transient transfection assays without selection.

BHK-21 cells were transiently transfected with an IgG expression construct [Geserick C et al. (2000). Enhanced productivity during controlled proliferation of BHK-21 cells in continuously perfused bioreactors. Biotech and Bioeng 69: 266-274] and the SEAP expression constructs indicated by using the calcium coprecipitation method. IgG ELISA and SEAP activities from cell culture supernatants were determined 2 days after transfection. The SEAP activity is given relative to the expression level of the original SEAP construct and an IgG standard [Geserick C et al. (2000). Enhanced productivity during controlled proliferation of BHK-21 cells in continuously perfused bioreactors. Biotech. and Bioeng. 69, 266-274].

FIG. 7. Fraction of SEAP expressing clones from stop codon suppression dependent translation constructs after selection.

BHK-21 cells were transfected using a stop codon suppression dependent neo reading frame translating construct (SEAPstopMneo) or cotransfection of separate SEAP and neo constructs at 1:10 (SEAP+1/10 neo) and 1:1 (SEAP+neo) ratios of plasmid DNA, respectively. SEAP activity from drug resistant BHK-21 cell single clones was determined by the agarose overlay technique (Kirchhoff, S. et al. (1995) Identification of mammalian cell clones exhibiting highly regulated expression from inducible promoters. Trends Genet. 11: 219-220; McCracken, A. A. et al. (1984) Studies on the secretion of serum proteins from rat hepatoma cells. Hepatology. 4: 715-721; Walls, J. D., Grinnell, B. W. (1990) A rapid and versatile method for the detection and isolation of mammalian cell lines secreting recombinant proteins. Biotechniques 8: 138-142). The number of SEAP positive staining cells is given relative to the total number of G418 resistant clones obtained.

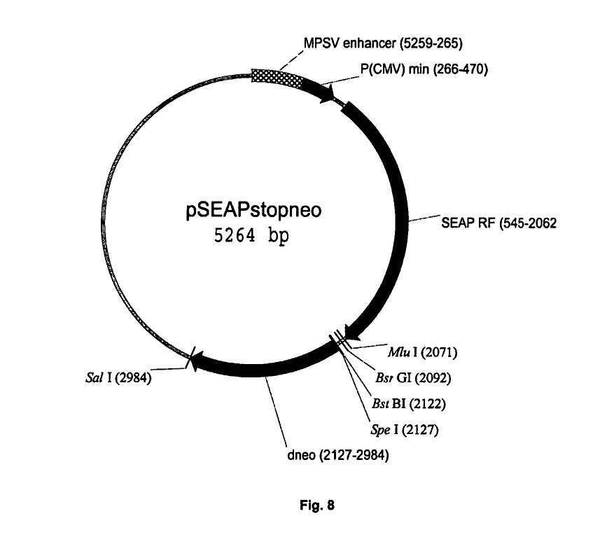

FIG. 8: Plasmid map of pSEAPstopneo

The promoter contains the minimal promoter sequence from the human CytoMegaloVirus promoter (CMV), the enhancer elements are derived from the myeloproliferative sarcoma virus promoter, sd-sa are a splice-donor and splice acceptor site from SV40 late splice site, SEAP (5452-2062) encodes the secreted form of the placental alkaline phosphatase, dneo (2271-3128) refers to a sequence encoding an N-terminally truncated neo fragment. The locations of these elements are given in the figure in parenthesis.

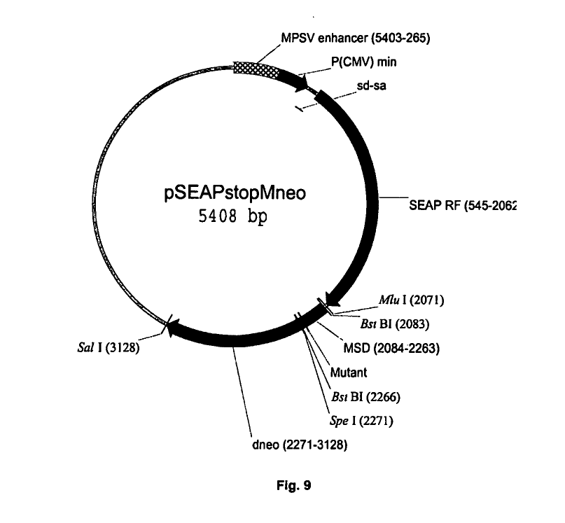

FIG. 9: Plasmid map of pSEAPstopMneo

MSD (2048-2263) encodes a membrane spanning domain derived from the platelet derived growth factor receptor (PDGFR), Mutant refers to the elimination of an non-initiating AUG codon that was present in the original PDGFR membrane spanning domain. The remaining elements are described in the legend of FIG. 8.

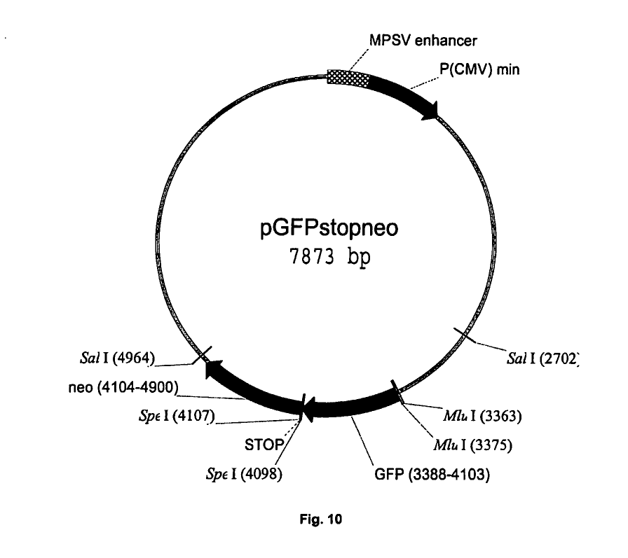

FIG. 10: Plasmid map of pGFPstopneo

GFP encodes a green fluorescent protein derived from the jellyfish Aequorea victoria (Tsien, R. Y. (1998) The green fluorescent protein. Annu. Rev. Biochem. 67: 509-544). The remaining elements are described in the legend of FIG. 8.

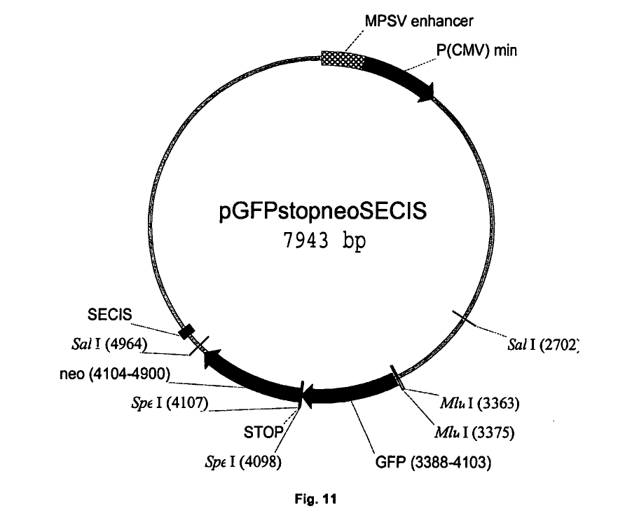

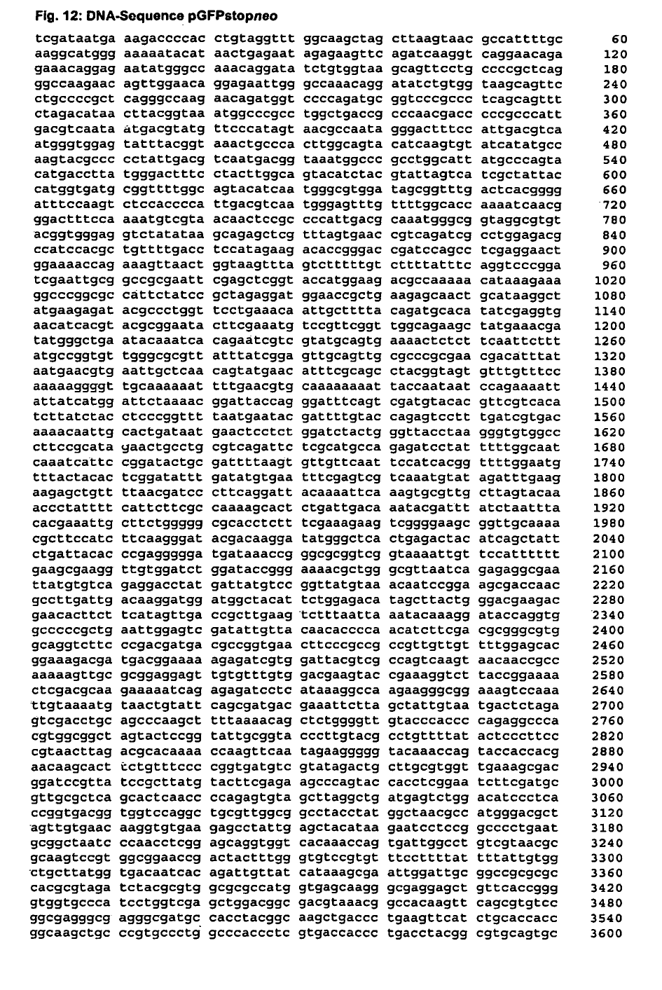

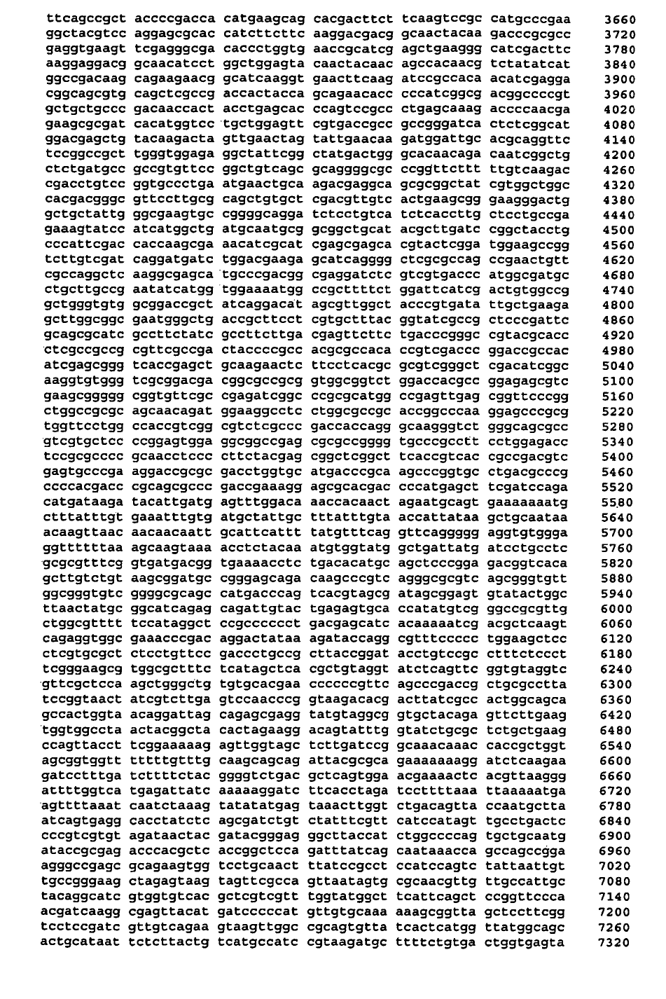

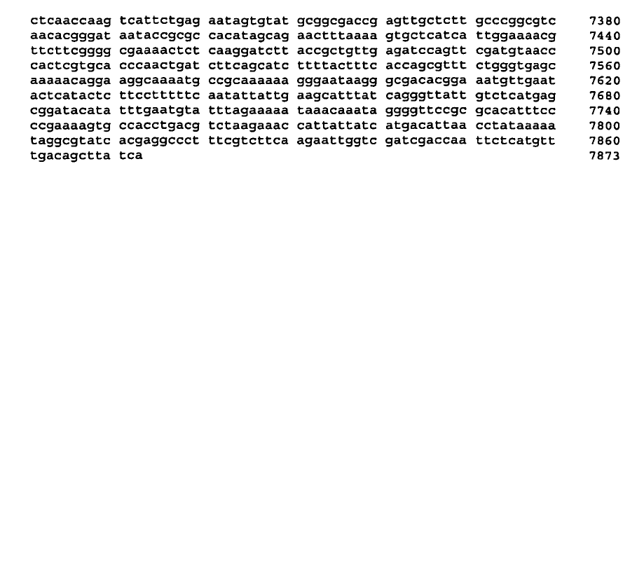

FIG. 11: Plasmid map of pGFPstopneoSECIS

SECIS refers to a selenocystein insertion element that promotes the translation of UGA codons [Walczak, R. et al. (1997) Solution structure of SECIS, the mRNA element required for eukaryotic selenocysteine insertion—interaction studies with the SECIS-binding protein SBP. Biomed. Environ Sci. 10: 177-181; Hubert, N. et al. (1996) RNAs mediating cotranslational insertion of selenocysteine in eukaryotic selenoproteins. Biochimie 78: 590-569]. The remaining elements are described in the previous figure legends.

FIG. 12: DNA-Sequence pGFPstopneo

All elements are described in the legend of FIG. 10.

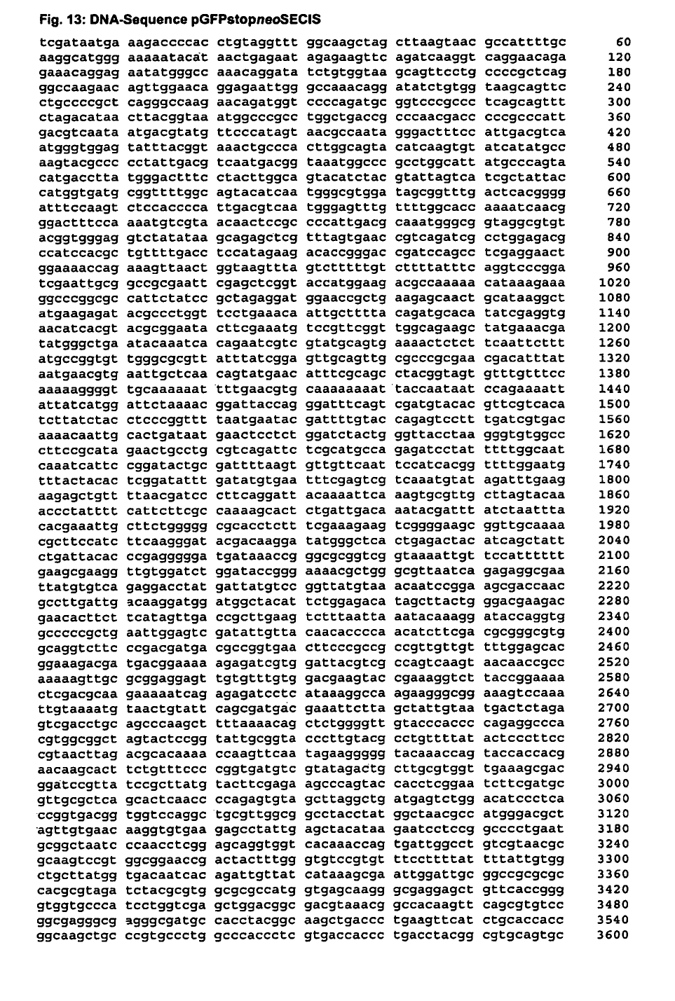

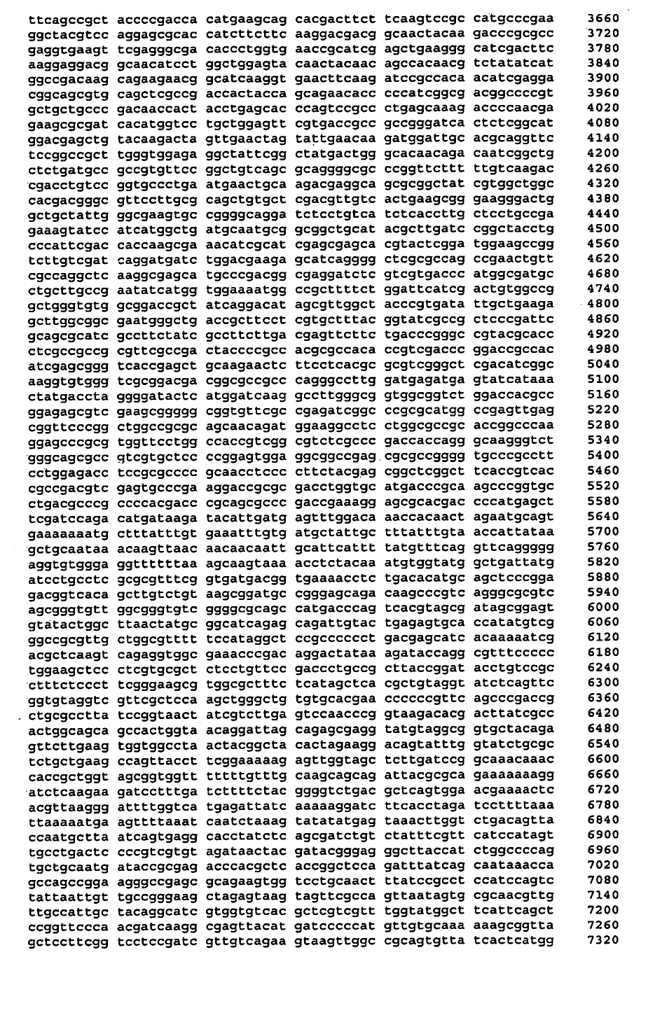

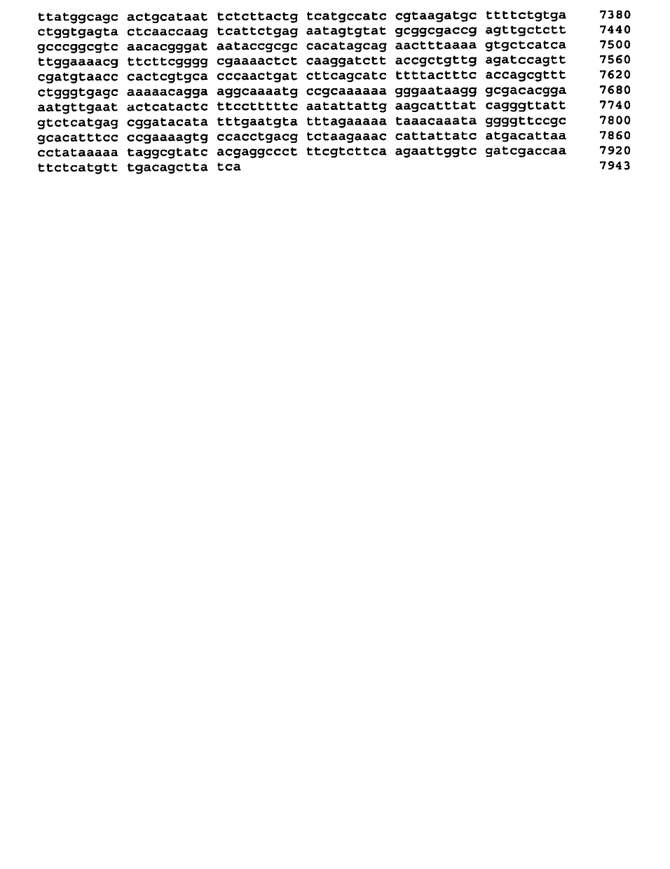

FIG. 13: DNA-Sequence pGFPstopneoSECIS

All elements are described in the legend of FIG. 11.

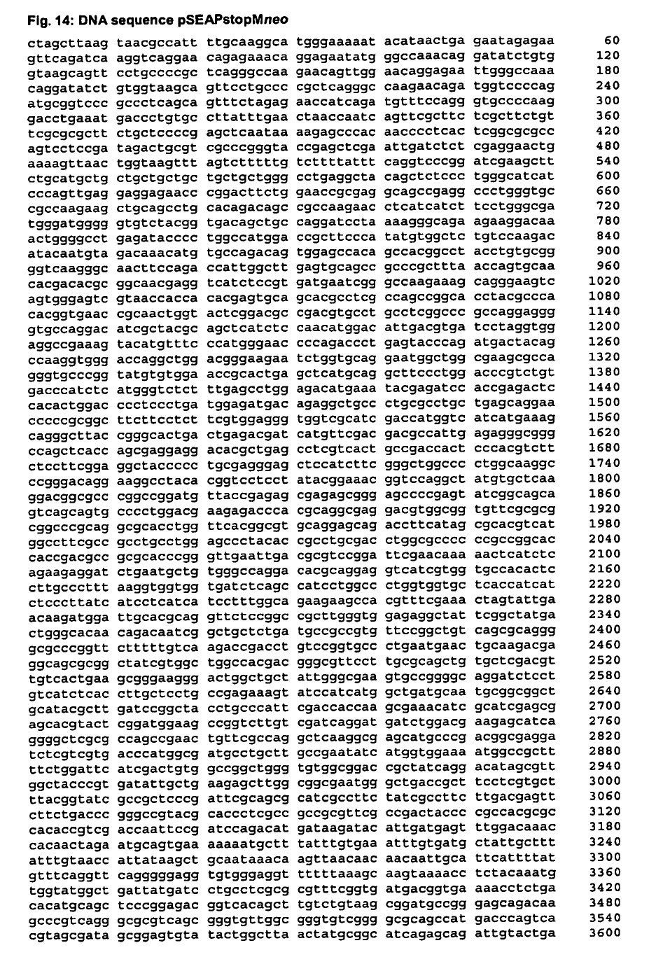

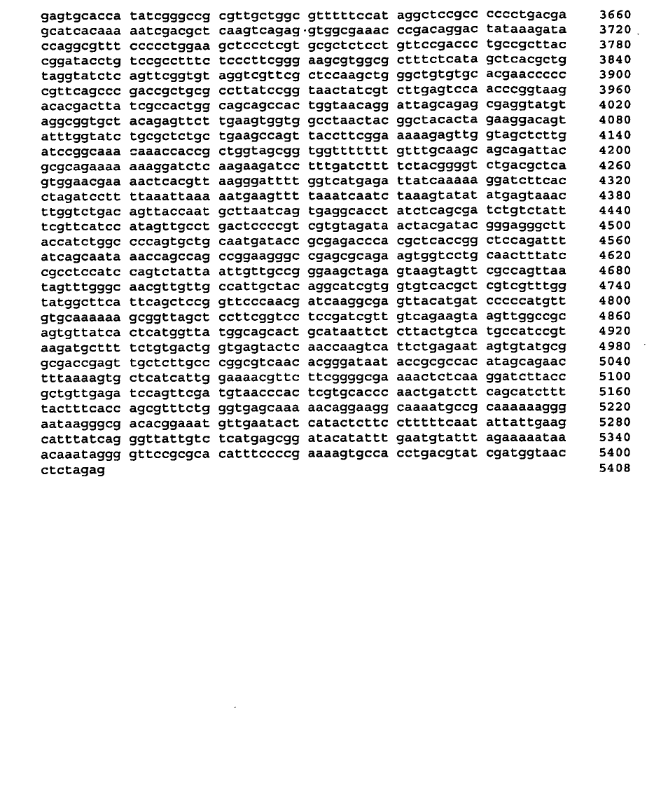

FIG. 14: DNA sequence pSEAPstopMneo

All elements are described in the legend of FIG. 9.

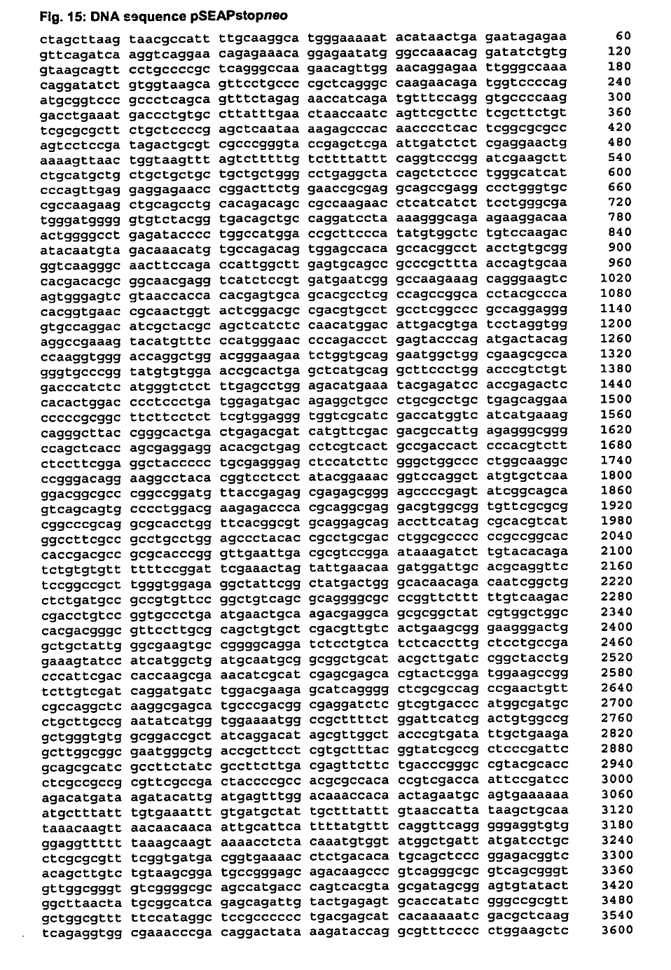

FIG. 15: DNA sequence pSEAPstopneo

All elements are described in the legend of FIG. 8.

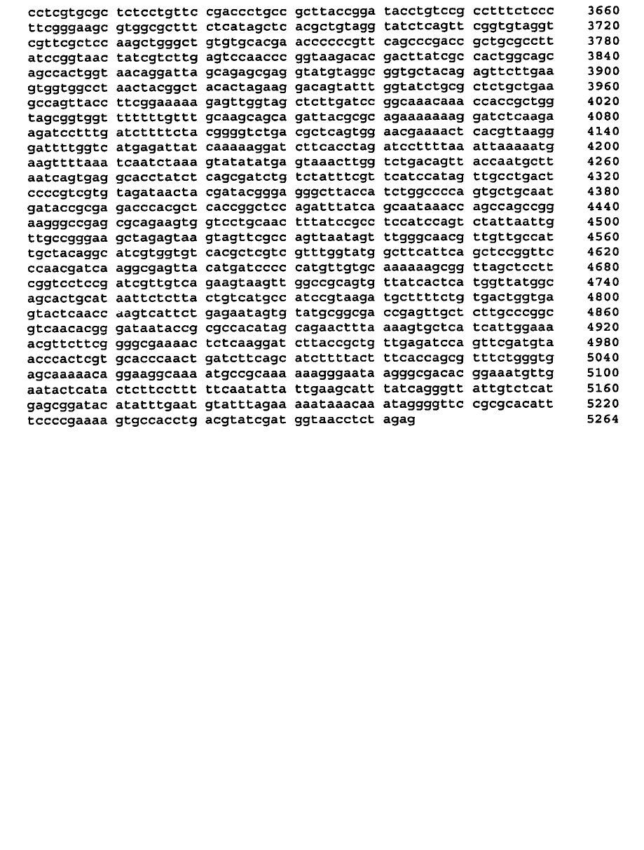

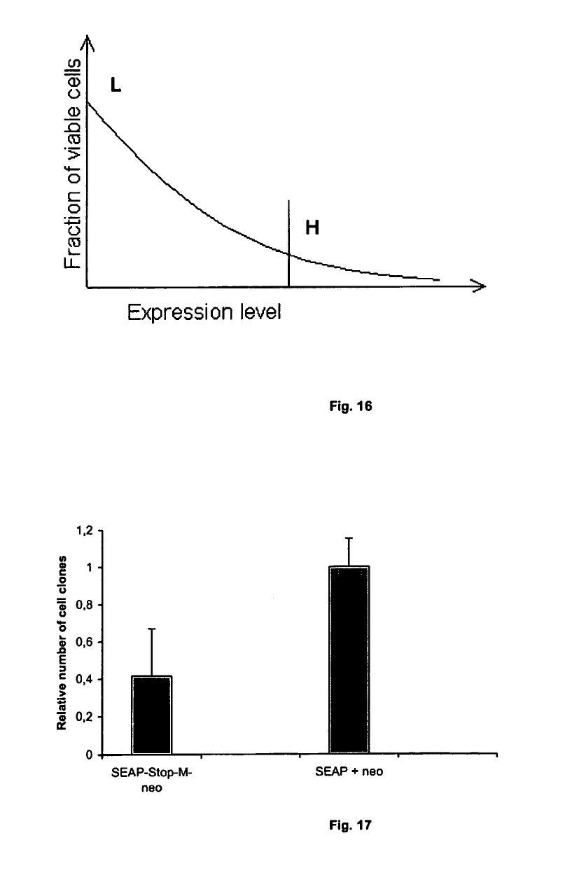

FIG. 16: Influence of selection pressure on the expression levels of producer cells

Schematic drawing depicting the statistically expected number of producer cells that survive low selection pressure (L) as area below the depicted curve. A fraction of cells with increased expression permits survival even at high selection pressures (H). High selection pressure reduces the screening effort to identify the small number of optimal producer cells (right) by preventing growth of inefficient producer cells (left).

FIG. 17: Reduced number of surviving clones

BHK-21 cells were transfected with recombinant plasmid DNA unsing calcium-phosphate co-precipitation (Wigler M, Silverstein S, Lee L S, Pellicer A, Cheng Y, Axel R. Transfer of purified herpes virus thymidine kinase gene to cultured mouse cells. Cell. 1977, 11:223-32). Two days after transfection 1 mg G418/ml cell culture medium was added and the cells incubated further until no surviving cells could detected in a control experiment. The bars indicate the fraction of single cell producer clones surviving the selection procedure. The numbers were calculated as avreage of two independent experiments (SEAP-Stop-M-neo, transfection with DNA of the plasmid pSEAPstopMneo; SEAP+neo, conventional cotransfection with a SEAP coding and neo coding plasmid DNA).

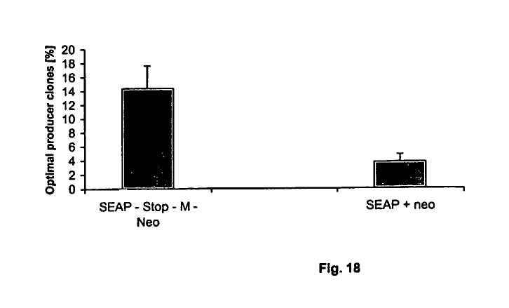

FIG. 18: Increased frequency of producer clones

The graph depicts the faction of single cell colonies that scored positive in a SEAP filter overlay assay (Kirchhoff S, Koster M, Wirth M, Schaper F, Gossen M. Bujard H, Hauser H. Identification of mammalian cell clones exhibiting highly regulated expression from inducible promoters. Trends Genet. 1995; 11:219-20.). The lowest expression level of a cell clone that scored positive in this test was 0,01 μg/cell. The experimental details are described in the FIG. 17.

### EXAMPLES

The following examples illustrate the invention, i.e. the feasibility of stop codon suppression-dependent fusion protein translation to improve the coupling of product and selectable marker gene expression.

As shown in FIG. 1, a selectable marker gene was expressed strictly dependent on the translation of a product gene whereby the initiation codon of the selectable marker gene neomycin phosphotransferase (neo) was removed by PCR-mediated mutagenesis thus leading to a truncated neo reading frame which was fused in frame to the complete product gene-reading frame including the termination codon. As examples for product genes the intracellular green fluorescent protein GFP derived from the bioluminescent jellyfish Aequorea victoria [Naylor L H. (1999) Reporter gene technology: the future looks bright. Biochem. Pharmacol. 58: 749-757; Keith, J., Frank, H., Martine, K. (1999) Lowing jellyfish, luminescence and a molecule called coelenterazine Trends in Biotechnology 12: 477-481; Ellenberg, J., Lippincott-Schwartz, J., Presley, J. F. (1999) Dual-color imaging with GFP variants. Trends Cell. Biol. 9: 52-56] or a secreted form of human alkaline phosphatase (SEAP) [Berger, J. et al. (1988) The secreted alkaline phosphatase: a powerful new quantitative indicator of gene expression in eukaryotic cells. Gene 66: 1-10] was used in various configurations (FIG. 1). DNA manipulations were performed according to Maniatis, T. et al. [Maniatis, T., Fritsch, E. F., Sambrook, J., (1982) In: Molecular cloning. A laboratory manual. Cold Spring Harbor Laboratory, Cold Spring Harbor, N.Y.]. Manipulations of BHK-21 host cells were performed according to Mueller P P et al. or Geserick C et al. [Mueller P P et al. (1999) Recombinant glycoprotein product quality in proliferation controlled BHK-21 cells. Biotech. and Bioeng. 65: 529-536 and Geserick, C. et al. (2000) Enhanced productivity during controlled proliferation of BHK-21 cells in continuously perfused bioreactors. Biotech and Bioeng 69: 266-274].

### Example 1

### Improved Coupling of Gene Expression by Stop Codon Suppression-Dependent Fusion Protein Translation

The GFPneo construct encoded a fusion protein with an N-terminal GFP domain and a C-terminal neo domain (FIG. 2A). When the GFP reading frame was terminated by a stop codon (GFPstopneo), the encoded GFP protein was expressed from this construct. Ribosomes have a small error rate resulting in C-terminally extended proteins (FIG. 2B). These naturally occurring translational error events were utilized to express minor amounts of GFPneo fusion protein (FIG. 2B).

This natural rate of mistranslation generated sufficient neomycin phosphotransferase activity to render transfected BHK-21 21 cells G418 resistant (see FIG. 3; GFPstopneo). The expected GFP-neo fusion protein confered neo activity and G418 resistance to the transfected cells. A construct (GFPneo) without in-frame stop codon at the end of the GFP reading frame was used in a parallel experiment to demonstrate that a GFP-neo fusion protein confers G418 resistance (Geserick, C. et al. (2000) Enhanced productivity during controlled proliferation of BHK-21 cells in continuously perfused bioreactors. Biotech. and Bioeng. 69: 266-274) (see FIG. 3).

In addition, a SECIS sequence has been added to the stop-codon suppression-dependent construct (GFPstopneoSECIS). All described constructs resulted in G418-resistant colonies, whereas mock transfected cells, i.e. cells that were treated identically except that the DNA was omitted in the procedure, remained G418 sensitive. This demonstrated that all constructs lead to the synthesis of sufficient gene products, most likely GFP-neo fusion proteins, with neo activity high enough to confer G418-resistance to transfected cells.

Limiting translation of the neo reading frame led to survival in the presence of G418 of only those transfected cell clones that showed high expression levels. The level of product formation in the resistant cell clones was determined by measuring the GFP fluorescence intensity. For this purpose, a cell suspension in PBS with 5% FCS was stained with 0.5 microgrammiml propidium iodide and analyzed with a FACScan, Beckton Dickinson (see FIG. 4). Fluorescence levels in cells transfected with the GFP stop-codon containing construct (see FIG. 4; GFPstopneo) were above those obtained from control constructs (GFPneo, or GFPstopneoSECIS). This demonstrates a limiting activity of the neo gene product that can advantageously be used to isolate high producer cell clones.

Stop codon dependent translational fusion provided a strict dependence of selectable marker gene expression on the expression of the gene of interest. Product formation was maintained even during prolonged growth in the presence of 1 mg/ml G418 in the culture medium (see FIG. 5). In contrast, expression of conventionally co-transfected constructs in the same cells has been shown to decrease with time [Geserick, C. et al. (2000) Enhanced productivity during controlled proliferation of BHK-21 cells in continuously perfused bioreactors. Biotech. and Bioeng. 69: 266-274].

### Example 2

### Secreted Product Protein—Stop Codon Suppression-Dependent Fusion Protein Translation

The selection system was applicable to secreted product proteins, e.g. secreted alkaline phosphatase (SEAP). SEAP activity from stop-codon dependent translated constructs (SEAPstopMneo, for pSEAPstopMneo see FIG. 8) was compared with a control (SEAP) in transient transfection assays. SEAP activity was determined according to Berger, J. et al. [Berger, J. et al. (1988) The secreted alkaline phosphatase: a powerful new quantitative indicator of gene expression in eukaryotic cells. Gene 66: 1-10] (see FIG. 6). The data showed that extension of the product mRNA with neo coding sequences did not impede SEAP expression efficiency in the absence of selection pressure. All constructs led to similar SEAP expression, indicating that the added neo sequences had no significant detrimental effect on expression levels.

The fraction of BHK-21 producer cells among clones surviving the selection in the presence of G418 [Geserick, C. et al. (2000) Enhanced productivity during controlled proliferation of BHK-21 cells in continuously perfused bioreactors. Biotech. and Bioeng. 69: 266-274] was a measure of the stringency of the coupling between product gene expression and selectable marker gene expression. The fraction of producer clones was determined after selection for survival and growth in the presence of G418 (see FIG. 7 and Tab. 1). The number of producer cell clones (FIG. 7 and Tab. 1, SEAPstopMneo) was significantly higher than in the control transfections (FIG. 7 and Tab. 1, SEAP+neo), suggesting that stop codon-dependent translation selection results in a superior coupling rate.

TABLE 1
Efficiency of clone formation from stop codon suppression
dependent translation constructs after selection.

| | Construct: | | |
|---|---|---|---|
| | SEAPstopMneo | SEAP + 1/10 neo | SEAP + neo |
| Number of clones | 255 | 27 | 394 |
| Number of producer | 93 | 3 | 14 |
| clones | | | |
| BHK-21 cells were transfected by the calcium phosphate coprecipitation method using a stop codon suppression dependent neo reading frame translating construct (SEAPstopMneo) or cotransfection of separate SEAP and neo constructs at 1:10 (SEAP + 1/10 neo) and 1:1 (SEAP + neo) | | | |
| # ratios of plasmid DNA, respectively. 1000 microgram per ml culture was added and the incubation continued until all cells from a non-transfected control culture were dead. Producer clones were determined by an agarose overlay diffusion filter binding assay for alkaline phosphatase activity. | | | |

### Example 3

### Construction of pGFPstopneo

The plasmid pGFPstopneo was constructed from an existing plasmid PMC2LUZI [Mielke, C., Tummler, M., Bode, J. A., simple assay for puromycin N-acetyltransferase: selectable marker and reporter. Trends Genet. (1995) 11: 258-259] by standard recombinant DNA techniques [Sambrook et al. (1989) In: Molecular Cloning: A Laboratory Manual. Cold Spring Harbor Laboratory Press, second edition].

The neo gene was derived from the bacterial transposon Tn 5. To create neo fusion proteins, the AUG initiation codon was deleted from the neo reading frame by PCR-mediated mutagenesis using the primers PMU212 5′-GCGGTCGACGGCCCGGGTCAGAAGAACTC-3′ and PMU213 5′-GGCACTAGTCACACCGGTGGATTGCACGCAGGTTCTCCGGCC-3′ and a fusion PCR was performed with eGFP (Clontech Laboratories, Inc., Palo Alto, Calif.) to generate the GFPstopneo reading frame fusion. The resulting PCR fragment was cleaved using the restriction endonucleases AscI and SmaI and ligated to the vector backbone in place of the PAC gene downstream of the IRES (Internal Ribosome Entry Site) element after cleavage by the same enzymes. The ligation mixture was transformed into competent E. coli cells (E. coli JM109) by electroporation. Transformants were selected on Ampicillin containing solid medium. DNA was isolated from individual colonies and tested for the presence of expected restriction fragments. The critical sequences surrounding the stop codon were confirmed by enzymatic sequencing [Sanger, F., Coulson, A. R. (1975) A rapid method for determining sequences in DNA by primed synthesis with DNA polymerase. J. Mol. Biol. 94: 441-448].

### Example 4

### Construction of pGFPstopneoSECIS

A SECIS consensus element was synthesized as two complementary oligonucleotides of the following

sequence (5′to 3′):
SECIS1: 5′-CAGGGCCTTGGATGAGATGAGTATCATAAACTATGACC-
TAGGGGATACTCATGGATCAAGGCCTTGGGC-3′
and
SECIS2: 5′-CCAAGGCCTTGATCCATGAGTATCCCCTAGGTCATAGT-
TTATGATACTCATCTCATCCAAGGCCCTGGGC-3′.

It was inserted downstream of the neo reading frame into the unique SacII restriction site of the plasmid pGFPstopneo by using T4 DNA ligase.

### Example 5

### Construction of pSEAPstopneo

pSEAPstopneo is a plasmid vector comprising the secreted alkaline phosphatase-reading frame followed the neo reading frame. The construct was derived from pSEAPstopMneo by cleavage with BstBI and religation according to Maniatis, T. et al. [Maniatis, T., Fritsch. E. F., Sambrook, J., (1982) In: Molecular cloning. A laboratory manual. Cold Spring Harbor Laboratory, Cold Spring Harbor, N.Y.].

### Example 6

### Construction of pSEAPstopMneo

pSEAPstopMneo is a plasmid vector comprising the secreted alkaline phosphatase-reading frame followed by a Stop codon and a membrane-spanning domain and by the neo reading frame. The construct was derived from the plasmid pMPSVHE [Artelt, P. et al. (1988) Vectors for efficient expression in mammalian fibroblastoid, myeloid and lymphoid cells via transfection or infection. Gene 68: 213-219) and the gene encoding the secreted form of alkaline placental phosphatase [Berger, J. et al. (1988) The secreted alkaline phosphatase: a powerful new quantitative indicator of gene expression in eukaryotic cells. Gene 66: 1-10). The membrane-spanning domain is derived by PCR amplification from the pHook-1™ vector (Invitrogen, Carlsbad, Calif.) using the two primers PMU202 and PMU203 and was inserted as a Mfel restriction fragment into the unique EcoRI site of BMS2, such that both sites were destroyed. The initiation codon deficient neo reading frame was inserted as a DNA fragment isolated after enzymatic cleavage of pGFPstopneo-SECIS DNA with SalI and SpeI, resulting in pSEAPstop(pA)Mneo. This plasmid DNA was then cut with BspEl such that the p(A) sequence was removed. The remaining plasmid DNA was religated, resulting in pSEAPstopMneo.

PMU202: 5′-CGCCAATTGGTCGACACTAGTTTCGAAACGTGGCTTCT-
TCTGCCAAAGGATGATGAGGA-3′
and
PMU203:
5′-GCGCAATTGACGCGTTGAACGCGTTCCGGAATAAATAAATCCGGAT-
TCGAACAAAAACTCATCTCAGAAG-3′

### Example 7

### Efficient Selection of Cell Clones Highly Expressing a Gene of Interest

The efficiency of recombinant gene expression after integration of a recombinant gene into the genome of a mammalian cell is dependent on a number of variables. Even if a selection marker is expressed in a way that the recombinant cell survives selection conditions, the expression of the selectable marker gene does not directly correlate with the expression level of the gene of interest. Selection procedures known from state of the art result in a large fraction of low producer cell clones that may express below 1 microgramm recombinant protein per 10 000 000 cells per day and in a small fraction of cell clones highly expressing a gene of interest, i.e. cells that express above 1 microgramm per 10 000 000 cells per day. These cell clones highly expressing a gene of interest can be identified only with a considerable screening effort. The screening effort can be reduced as follows: (1) Increasing the selection pressure to eliminate low producer cells and thus to reduce the number of cell clones that must be screened, and (2) establishing a tight link between the expression of the selectable marker gene and the expression of the gene of interest to avoid the isolation of clones that efficiently express only the selectable marker gene and not the gene of interest. As a consequence, the average expression level of the cell clones surviving selection conditions will be higher (FIG. 16).

To demonstrate that a recombinant gene expression vector as disclosed herein can be used in a process for efficiently selecting a host cell clone highly expressing said gene of interest, pSEAPstopMneo plasmid DNA was transfected into BHK-21 cells unsing calcium-phosphate co-precipitation (Wigler M, Silverstein S, Lee L S, Pellicer A, Cheng Y, Axel R. Transfer of purified herpes virus thymidine kinase gene to cultured mouse cells. Cell. 1977, 11: 223-32). Two days after transfection, selection conditions were applied by adding 1 mg/ml G418 to cell culture medium. Cells were incubated under said selection conditions until no living cells could be detected in a control experiment (cells without a selectable marker gene were also incubated in cell culture medium containing 1 mg/ml G418). In a parallel experiment, cells were co-transfected with (1) a SEAP encoding plasmid DNA construct and (2) a neo plasmid DNA construct according to a conventional co-transfection procedure. As a result, the number of surviving cell clones obtained with the pSEAPstopMneo plasmid DNA was reduced by at least 50% indicating that pSEAPstopMneo can be used to reduce the screening effort (FIG. 17).

To demonstrate that the average expression rate of the gene of interest is higher in cell clones comprising pSEAPstopMneo, approximately 100 cell clones obtained with the above mentioned experiment were scored in a SEAP filter overlay assay (Kirchhoff S, Koster M, Wirth M, Schaper F, Gossen M, Bujard H, Hauser H. Identification of mammalian cell clones exhibiting highly regulated expression from inducible promoters. Trends Genet. 1995; 11: 219-20). As a result. 14% of cell clones transfected with pSEAPstopMneo highly expressed the gene of interest which was three times more than then number of highly expressing cell clones obtained from co-transfecting BHK-21 cells with a SEAP encoding plasmid DNA construct and a neo plasmid DNA construct (FIG. 18). This result clearly demonstrates the use of a recombinant gene expression vector—as disclosed herein—in a process to efficiently selecting host cell clones highly expressing a gene of interest.

1. A recombinant gene expression vector comprising in the following order a promoter sequence, a gene of interest, a translational stop signal and translationally linked to said gene of interest a selectable marker gene whereby a recombinant gene expression vector is excluded which recombinant gene expression vector contains in the following order a promoter sequence, a gene encoding β-galactosidase, a TGA stop codon, and translationally linked to said β-galactosidase gene a gene encoding luciferase.

2. A recombinant gene expression vector as claimed in claim 1, comprising at least two genes of interest.

3. A recombinant gene expression vector according to claim 1, wherein said selectable marker gene encodes a functional protein for selection of host cells comprising said recombinant gene expression vector and expressing said selectable marker gene.

4. A recombinant gene expression vector according to claim 1, wherein said translational stop signal is at least one stop codon selected from the group of TAA, TGA and TAG.

5. A recombinant gene expression vector according to claim 1, comprising downstream of said selectable marker gene a SECIS element which modulates the expression of said selectable marker gene from the corresponding mRNA.

6. A recombinant gene expression vector, comprising in the following order a promoter sequence, a gene of interest, a translational stop signal and translationally linked to said gene of interest a selectable marker gene wherein the gene of interest encoding a secreted product protein and the selectable marker gene are separated by a translational stop signal and an in frame stop transfer sequence.

7. A host cell comprising a recombinant gene expression vector as claimed in claims 1 to 6, which host cell is capable of expressing both said gene of interest and as a fusion protein said gene of interest and said selectable marker gene.

8. A host cell as claimed in claim 7 which is a host cell line and which is stably maintaining the expressing of said gene of interest during cultivation.

9. A process for producing a host cell clone comprising a recombinant gene expression vector as claimed in claims 1 to 6, which process comprises transforming a host cell with said recombinant gene expression vector as claimed in claims 1 to 6 and selecting a host cell clone comprising said recombinant gene expression vector on the expression of said selectable marker gene.

10. A process for producing a protein encoded by a gene of interest, which process comprises the production of a host cell line comprising a recombinant gene expression vector, the cultivation of said host cell line, stably maintaining the expression of said gene of interest, and recovery of the product of said gene of interest, whereby said recombinant gene expression vector comprises in the following order a promoter sequence, a gene of interest, a translational stop signal and translationally linked to said gene of interest a selectable marker gene, and whereby the production of said host cell line comprises the transformation with said recombinant gene expression vector and the selection of a host cell comprising said recombinant gene expression vector on the expression of said selectable marker gene.

11. A process for producing a secreted product protein encoded by a gene of interest, which process comprises the production of a host cell line comprising a recombinant gene expression vector, the cultivation of said host cell line, stably maintaining the expression of said gene of interest, and recovery of said secreted product protein from the cell's surrounding culture medium, whereby said recombinant gene expression vector comprises in the following order a promoter sequence, a gene of interest encoding a secreted product protein, a translational stop signal, an in frame stop transfer sequence and translationally linked to said gene of interest a selectable marker gene, and whereby the production of said host cell line comprises the transformation with said recombinant gene expression vector and the selection of a host cell comprising said recombinant gene expression vector on the expression of said selectable marker gene.

12. Use of a recombinant gene expression vector as claimed in claims 1 to 6 in a process for efficiently selecting a host cell clone highly expressing a gene of interest.

13. A process of producing a host cell line highly expressing a gene of interest, which process comprises a process for producing a host cell clone as claimed in claim 9 and efficiently selecting a host cell clone highly expresses said gene of interest.


SEQUENCE LISTING (10 sequences)

SEQ ID NO: 1 - DNA, 29 bp, Artificial
  PMU212 is a primer used in vitro for PCR amplification of the neo gene.
gcggtcgacggcccgggtcagaagaactc

SEQ ID NO: 2 - DNA, 42 bp, Artificial
  PMU213 is a primer used in vitro for PCR amplification of the neo gene.
ggcactagtcacaccggtggattgcacgcaggttctccggcc

SEQ ID NO: 3 - DNA, 69 bp, Artificial
  SECIS1 is used as one of two complementary oligonucleotides for synthesis of the SECIS consensus element of pGFPstopneoSECIS
cagggccttggatgagatgagtatcataaactatgacctaggggatactcatggatcaaggccttgggc

SEQ ID NO: 4 - DNA, 70 bp, Artificial
  SECIS2 is used as one of two complementary oligonucleotides for synthesis of the SECIS consensus element of pGFPstopneoSECIS
ccaaggccttgatccatgagtatcccctaggtcatagtttatgatactcatctcatccaaggccctgggc

SEQ ID NO: 5 - DNA, 59 bp, Artificial
  PMU202 is a primer used in vitro for PCR amplification of the membrane spanning domain from pHook-1(TM) vector for generation of pSEAPstopMneo
cgccaattggtcgacactagtttcgaaacgtggcttcttctgccaaaggatgatgagga

SEQ ID NO: 6 - DNA, 70 bp, Artificial
  PMU203 is a primer used in vitro for PCR amplification of the membrane spanning domain from pHook-1(TM) vector for generation of pSEAPstopMneo
gcgcaattgacgcgttgaacgcgttccggaataaataaatccggattcgaacaaaaactcatctcagaag

SEQ ID NO: 7 - DNA, 7873 bp, Artificial
  pGFPstopneo is a circular Plasmid DNA
tcgataatgaaagaccccacctgtaggtttggcaagctagcttaagtaacgccattttgcaaggcatgggaaaaatacataactgagaatagagaagttcagatcaaggtcaggaacagagaaacaggagaatatgggccaaacaggatatctgtggtaagcagttcctgccccgctcagggccaagaacagttggaacaggagaattgggccaaacaggatatctgtggtaagcagttcctgccccgctcagggccaagaacagatggtccccagatgcggtcccgccctcagcagtttctagacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcgaggaactggaaaaccagaaagttaactggtaagtttagtctttttgtcttttatttcaggtcccggatcgaattgcggccgcgaattcgagctcggtaccatggaagacgccaaaaacataaagaaaggcccggcgccattctatccgctagaggatggaaccgctgaagagcaactgcataaggctatgaagagatacgccctggttcctgaaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctacggtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatcttatctacctcccggttttaatgaatacgattttgtaccagagtcctttgatcgtgacaaaacaattgcactgataatgaactcctctggatctactgggttacctaagggtgtggcccttccgcatagaactgcctgcgtcagattctcgcatgccagagatcctatttttggcaatcaaatcattccggatactgcgattttaagtgttgttcaattccatcacggttttggaatgtttactacactcggatatttgatatgtgaatttcgagtcgtcaaatgtatagatttgaagaagagctgttttaacgatcccttcaggattacaaaattcaaagtgcgttgcttagtacaaaccctattttcattcttcgccaaaagcactctgattgacaaatacgatttatctaatttacacgaaattgcttctgggggcgcacctctttcgaaagaagtcggggaagcggttgcaaaacgcttccatcttcaagggatacgacaaggatatgggctcactgagactacatcagctattctgattacacccgagggggatgataaaccgggcgcggtcggtaaaattgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggataccaggtggcccccgctgaattggagtcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgccaaaaagttgcgcggaggagttgtgtttgtggacgaagtaccgaaaggtcttaccggaaaactcgacgcaagaaaaatcagagagatcctcataaaggccaagaagggcggaaagtccaaattgtaaaatgtaactgtattcagcgatgacgaaattcttagctattgtaatgactctagagtcgacctgcagcccaagcttttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattgcggccgcgcgccacgcgtagatctacgcgtggcgcgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagactagttgaactagtattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatgagcttcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgatcgaccaattctcatgtttgacagcttatca

SEQ ID NO: 8 - DNA, 7943 bp, Artificial
  pGFPstopneoSECIS is a circular Plasmid DNA
tcgataatgaaagaccccacctgtaggtttggcaagctagcttaagtaacgccattttgcaaggcatgggaaaaatacataactgagaatagagaagttcagatcaaggtcaggaacagagaaacaggagaatatgggccaaacaggatatctgtggtaagcagttcctgccccgctcagggccaagaacagttggaacaggagaattgggccaaacaggatatctgtggtaagcagttcctgccccgctcagggccaagaacagatggtccccagatgcggtcccgccctcagcagtttctagacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcgaggaactggaaaaccagaaagttaactggtaagtttagtctttttgtcttttatttcaggtcccggatcgaattgcggccgcgaattcgagctcggtaccatggaagacgccaaaaacataaagaaaggcccggcgccattctatccgctagaggatggaaccgctgaagagcaactgcataaggctatgaagagatacgccctggttcctgaaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctacggtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatcttatctacctcccggttttaatgaatacgattttgtaccagagtcctttgatcgtgacaaaacaattgcactgataatgaactcctctggatctactgggttacctaagggtgtggcccttccgcatagaactgcctgcgtcagattctcgcatgccagagatcctatttttggcaatcaaatcattccggatactgcgattttaagtgttgttcaattccatcacggttttggaatgtttactacactcggatatttgatatgtgaatttcgagtcgtcaaatgtatagatttgaagaagagctgttttaacgatcccttcaggattacaaaattcaaagtgcgttgcttagtacaaaccctattttcattcttcgccaaaagcactctgattgacaaatacgatttatctaatttacacgaaattgcttctgggggcgcacctctttcgaaagaagtcggggaagcggttgcaaaacgcttccatcttcaagggatacgacaaggatatgggctcactgagactacatcagctattctgattacacccgagggggatgataaaccgggcgcggtcggtaaaattgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggataccaggtggcccccgctgaattggagtcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgccaaaaagttgcgcggaggagttgtgtttgtggacgaagtaccgaaaggtcttaccggaaaactcgacgcaagaaaaatcagagagatcctcataaaggccaagaagggcggaaagtccaaattgtaaaatgtaactgtattcagcgatgacgaaattcttagctattgtaatgactctagagtcgacctgcagcccaagcttttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattgcggccgcgcgccacgcgtagatctacgcgtggcgcgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagactagttgaactagtattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcccagggccttggatgagatgagtatcataaactatgacctaggggatactcatggatcaaggccttgggcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatgagcttcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgatcgaccaattctcatgtttgacagcttatca

SEQ ID NO: 9 - DNA, 5408 bp, Artificial
  pSEAPstopMneo is a circular Plasmid DNA
ctagcttaagtaacgccattttgcaaggcatgggaaaaatacataactgagaatagagaagttcagatcaaggtcaggaacagagaaacaggagaatatgggccaaacaggatatctgtggtaagcagttcctgccccgctcagggccaagaacagttggaacaggagaattgggccaaacaggatatctgtggtaagcagttcctgccccgctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtaccgagctcgaattgatctctcgaggaactgaaaagttaactggtaagtttagtctttttgtcttttatttcaggtcccggatcgaagcttctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtcgtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttgaattgacgcgtccggattcgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatcctttggcagaagaagccacgtttcgaaactagtattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacccgggccgtacgcaccctcgccgccgcgttcgccgactaccccgccacgcgccacaccgtcgaccaattccgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtatcgatggtaacctctagag

SEQ ID NO: 10 - DNA, 5264 bp, Artificial
  pSEAPstopneo is a circular Plasmid DNA
ctagcttaagtaacgccattttgcaaggcatgggaaaaatacataactgagaatagagaagttcagatcaaggtcaggaacagagaaacaggagaatatgggccaaacaggatatctgtggtaagcagttcctgccccgctcagggccaagaacagttggaacaggagaattgggccaaacaggatatctgtggtaagcagttcctgccccgctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtaccgagctcgaattgatctctcgaggaactgaaaagttaactggtaagtttagtctttttgtcttttatttcaggtcccggatcgaagcttctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtcgtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttgaattgacgcgtccggaataaagatcttgtacacagatctgtgtgttttttccggattcgaaactagtattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacccgggccgtacgcaccctcgccgccgcgttcgccgactaccccgccacgcgccacaccgtcgaccaattccgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtatcgatggtaacctctagag